(19) United States
(12) Patent Application Publication
(10) Pub. No.: US 2026/0007458 A1
(43) Pub. Date: Jan. 8, 2026

(54) DEVICES AND METHODS FOR BASIVERTEBRAL NERVE REMOVAL

(71) Applicant: KIC VENTURES, LLC, BURLINGTON, MA (US)

(72) Inventors: VITO LORE, BURLINGTON, MA (US); JASON SEALE, BURLINGTON, MA (US); JOSHUA FINKEL-LOPEZ, BURLINGTON, MA (US); KINGSLEY R. CHIN, BURLINGTON, MA (US); ERIK SPAYDE, BURLINGTON, MA (US); WILLIAM COSTIGAN, BURLINGTON, MA (US); RAJ KRISHNAN, BURLINGTON, MA (US)

(73) Assignee: KIC VENTURES, LLC, BURLINGTON, MA (US)

(21) Appl. No.: 19/259,780

(22) Filed: Jul. 3, 2025

Related U.S. Application Data
(60) Provisional application No. 63/668,681, filed on Jul. 8, 2024.

(51) Int. Cl. A61B 18/14; A61B 17/16; A61B 17/17; A61B 17/34; A61B 18/00; A61B 90/00; A61F 2/28; A61F 2/46

(52) U.S. Cl. CPC A61B 18/148; A61F 2/4601; A61B 17/1671; A61B 17/1757; A61B 17/3403; A61B 17/3423; A61B 17/3472; A61B 17/3496; A61B 2018/0044; A61B 2018/00577; A61B 2090/062; A61B 2090/3762; A61B 2090/3966; A61F 2002/2835; A61F 2310/00329; A61F 2310/00359; A61F 2310/00994

(57) ABSTRACT

A method for removing a basivertebral nerve (BVN), includes the following sequential steps. First, inserting a bone needle into a vertebral body of a vertebra and advancing the bone needle under fluoroscopy to a BVN base site. The bone needle includes an elongated shaft having a cannula extending from a proximal end to a distal end of the shaft, and a removable bevel tipped inner stylet extending through the cannula. Next, removing the inner stylet from the cannula and inserting a drill/awl through the cannula into the BVN base site and once the drill/awl position and depth is confirmed via fluoroscopy drilling the BVN base out. Next, removing the drill/awl from the cannula and inserting a cup curette through the cannula into the BVN base site to further resect under fluoroscopy the BVN base. Next, removing the cup curette from the cannula and inserting a pituitary rongeur through the cannula into the BVN base site and removing under fluoroscopy all resected portions of the BVN base. Next, removing the pituitary rongeur from the cannula and inserting an electric ablation probe through the cannula into the BVN base site and once the ablation probe depth and position is confirmed ablating and cauterizing any remaining BVN base and forming a lesion in the BVN site. Next, removing the ablation probe from the cannula and inserting a nerve monitoring probe through the cannula into the BVN base site and testing to ensure that the BVN base has been fully removed from the BVN site and a void has been formed. Next, removing the nerve monitoring probe from the cannula and attaching a syringe filled with bone graft to the proximal end of the shaft, and filling the void formed in the BVN site with bone graft.

### CROSS REFERENCE TO RELATED CO-PENDING APPLICATIONS

This application claims the benefit of U.S. provisional application Ser. No. 63/668,681 filed Jul. 8, 2024 and entitled “DEVICES AND METHODS FOR BASIVERTEBRAL NERVE REMOVAL”, the contents of which are expressly incorporated herein by reference.

### FIELD OF THE INVENTION

The present invention relates to devices and methods for basivertebral nerve (BVN) removal, and in particular, to devices, instruments, and methods that limit the size of the incision, and provide safe and accurate targeting and removal of the BVN.

### BACKGROUND OF THE INVENTION

The human spine is comprised of individual vertebrae 30 that are connected to each other to form a spinal column 29, shown in FIG. 1A. Referring to FIGS. 1B, FIG. 1C, FIG. 1D, and FIG. 1E, each vertebra 30 has a cylindrical bony body (vertebral body) 32, three winglike projections (two transverse processes 33, 35 and one spinous process 34), left and right facet joints 46, lamina 47, left and right pedicles 48 and a bony arch (neural arch) 36. Each vertebral body 32 has top and bottom endplates 32a, 32b, respectively. Within each vertebral body 32, the basivertebral nerve 52 is located, as shown in FIG. 1D and FIG. 1E. The basivertebral nerve 52 transmits pain signals from the vertebral endplates 32a, 32b to the brain, when the vertebral endplates are damaged. The bodies of the vertebrae 32 are stacked one on top of the other and form the strong but flexible spinal column 29. The neural arches 36 are positioned so that the space they enclose forms a tube, i.e., the spinal canal 37. The spinal canal 37 houses and protects the spinal cord and other neural elements. A fluid-filled protective membrane, the dura 38, covers the contents of the spinal canal. The spinal column is flexible enough to allow the body to twist and bend, but sturdy enough to support and protect the spinal cord and the other neural elements. The vertebrae 30 are separated and cushioned by thin pads of tough, resilient fiber known as inter-vertebral discs 40. There is a small opening (foramen) 42 between each vertebra 30, through which nerves 44 pass and go to different body parts. When the vertebrae are properly aligned the nerves 44 pass through without a problem. However, when the vertebrae are misaligned or a constriction 45 is formed in the spinal canal, the nerves get compressed 44a and may cause low back pain, leg pain or other neurological disorders. In addition to these structural causes of low back pain, disorders of the vertebral endplates 32a, 32b, such as, inflammation, fissuring, post-traumatic degeneration and intraosseous edema are believed to also contribute to low back pain.

In some of these pathologic circumstances of the vertebral endplates, basiverterbal nerve (BVN) ablation is performed to relieve the patient of discomfort. Safe and accurate targeting of the BVN is critical to the success of the ablation procedure. Accordingly, there is a need for devices, instruments, and methods that limit the size of the incision, and provide safe and accurate targeting of the BVN.

### SUMMARY OF THE INVENTION

The present invention relates to devices and methods for BVN removal, and in particular, to devices, instruments, and methods that limit the size of the incision, and provide safe and accurate targeting and removal of the BVN.

In general, in one aspect, the invention features a method for removing a basivertebral nerve (BVN), including the following sequential steps. First, inserting a bone needle into a vertebral body of a vertebra and advancing the bone needle under fluoroscopy to a BVN base site. The bone needle includes an elongated shaft having a cannula extending from a proximal end to a distal end of the shaft, and a removable bevel tipped inner stylet extending through the cannula. Next, removing the inner stylet from the cannula and inserting a drill/awl through the cannula into the BVN base site and once the drill/awl position and depth is confirmed via fluoroscopy drilling the BVN base out. Next, removing the drill/awl from the cannula and inserting a cup curette through the cannula into the BVN base site to further resect under fluoroscopy the BVN base. Next, removing the cup curette from the cannula and inserting a pituitary rongeur through the cannula into the BVN base site and removing under fluoroscopy all resected portions of the BVN base. Next, removing the pituitary rongeur from the cannula and inserting an electric ablation probe through the cannula into the BVN base site and once the ablation probe depth and position is confirmed ablating and cauterizing any remaining BVN base and forming a lesion in the BVN site. Next, removing the ablation probe from the cannula and inserting a nerve monitoring probe through the cannula into the BVN base site and testing to ensure that the BVN base has been fully removed from the BVN site and a void has been formed. Next, removing the nerve monitoring probe from the cannula and attaching a syringe filled with bone graft to the proximal end of the shaft, and filling the void formed in the BVN site with bone graft.

Implementations of this aspect of the invention may include one or more of the following features. The method further includes the following sequential steps. Removing the syringe and adjusting the bone needle position so that it is directed towards a superior vertebral endplate site of the vertebral body on a contra-lateral side of the vertebra. Next, reinserting the inner stylet into the cannula, advancing it to an edge of the superior vertebral endplate site and confirming the inner stylet position and depth with fluoroscopy. Next, removing the inner stylet from the cannula and reinserting the drill/awl through the cannula into the edge of the superior vertebral endplate site and once the drill/awl position and depth is confirmed via fluoroscopy drilling the edge of the superior vertebral endplate out.

Next, removing any tissue fragments from the edge of the superior vertebral endplate and reattaching the syringe filled with bone graft to the proximal end of the shaft, and filling a void formed in the edge of the superior vertebral endplate site with bone graft. Next, retracting the bone needle till it reaches an intersection between the vertebral body and a pedicle of the vertebra and filling a void formed by the retracted bone needle in the vertebral body with bone graft. Finally, removing the bone needle and suturing the bone needle insertion site. The bone graft is hydrated using a blood saline prior to filling the BVN void. The bone needle further comprises a handle attached to the proximal end of the shaft and wherein the syringe is connected to a proximal luer lock on the handle. The method further includes adding a contrast agent to the bone graft to visualize the filling of the BVN void with bone graft under fluoroscopy. The bone needle further includes depth markings on an outer surface of the elongated shaft. The cannula has a diameter of 3 mm and allows for multiple instruments to be inserted through the cannula for accessing sites in the vertebral body. The ablation probe is connected to a RF generator that generates electric current at a tip of the ablation probe. The bone needle, drill, cup curette, pituitary rongeur are made of stainless steel. The bone graft is a nano-fuse bioactive glass bone graft that comprises 45S5 bioactive glass with demineralized bone matrix (DBM), both coated with gelatin.

In general, in another aspect, the invention features a system for removing a basivertebral nerve (BVN), including the following. A bone needle for inserting into a vertebral body of a vertebra and advancing under fluoroscopy to a BVN base site. The bone needle includes an elongated shaft having a cannula extending from a proximal end to a distal end of the shaft, and a removable bevel tipped inner stylet extending through the cannula. A drill/awl for inserting through the cannula into the BVN base site and once the drill/awl position and depth is confirmed via fluoroscopy for drilling the BVN base out. A cup curette for inserting through the cannula into the BVN base site to further resect under fluoroscopy the BVN base. A pituitary rongeur for inserting through the cannula into the BVN base site and for removing under fluoroscopy all resected portions of the BVN base. An electric ablation probe for inserting through the cannula into the BVN base site and once the ablation probe depth and position is confirmed for ablating and cauterizing any remaining BVN base and forming a lesion in the BVN site. A nerve monitoring probe for inserting through the cannula into the BVN base site and for testing to ensure that the BVN base has been fully removed from the BVN site and a void has been formed. A syringe filled with bone graft for attaching to a proximal end of the cannula, and for filling the void formed in the BVN site with bone graft. A fluoroscope and/or a CT scanner.

The details of one or more embodiments of the invention are set forth in the accompanying drawings and description below. Other features, objects, and advantages of the invention will be apparent from the following description of the preferred embodiments, the drawings, and the claims.

### BRIEF DESCRIPTION OF THE DRAWINGS

Referring to the figures, wherein like numerals represent like parts throughout the several views:

FIG. 1A is a side view of the human spinal column;

FIG. 1B is an enlarged view of area A of FIG. 1A;

FIG. 1C is an axial cross-sectional view of a lumbar vertebra;

FIG. 1D is an enlarged view of area 50 of FIG. 1A;

FIG. 1E is a cross-sectional view of the vertebral body depicting the location of the BVN;

FIG. 2A-FIG. 2C depict Modic I, Modic II and Modic III type pathologies of the vertebral endplates;

FIG. 3A-FIG. 3B depict step 1 of the BVN removal procedure, according to this invention;

FIG. 4A-FIG. 4B depict step 2 of the BVN removal procedure, according to this invention;

FIG. 5A-FIG. 5B depict step 3 of the BVN removal procedure, according to this invention;

FIG. 6A-FIG. 6B depict step 4 of the BVN removal procedure, according to this invention;

FIG. 7A-FIG. 7B depict step 5 of the BVN removal procedure, according to this invention;

FIG. 8A-FIG. 8B depict step 6 of the BVN removal procedure, according to this invention;

FIG. 9A-FIG. 9B depict step 7 of the BVN removal procedure, according to this invention;

FIG. 10A-FIG. 10B depict step 8 of the BVN removal procedure, according to this invention;

FIG. 11A-FIG. 11B depict step 9 of the BVN removal procedure, according to this invention;

FIG. 12A-FIG. 12B depict step 10 of the BVN removal procedure, according to this invention;

FIG. 13A-FIG. 13B depict a flow diagram of the BVN removal procedure, according to this invention.

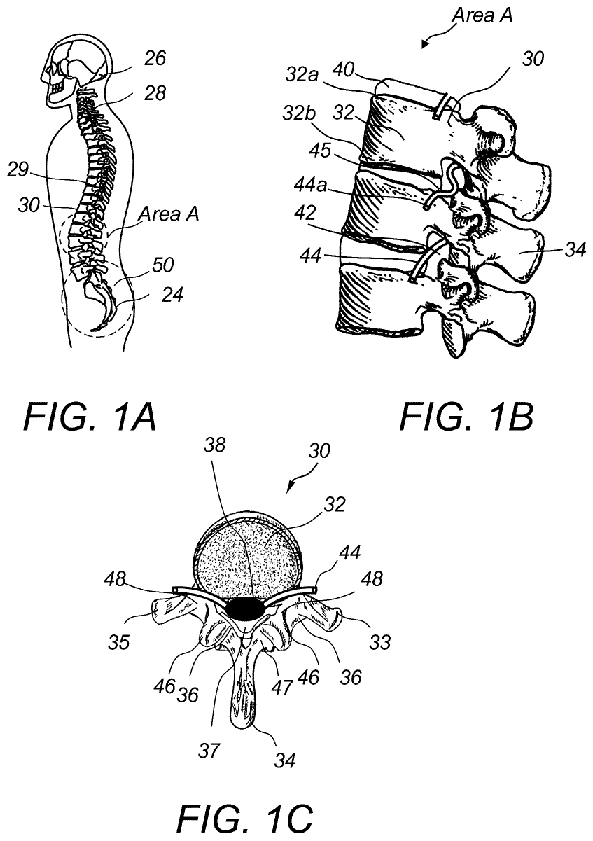

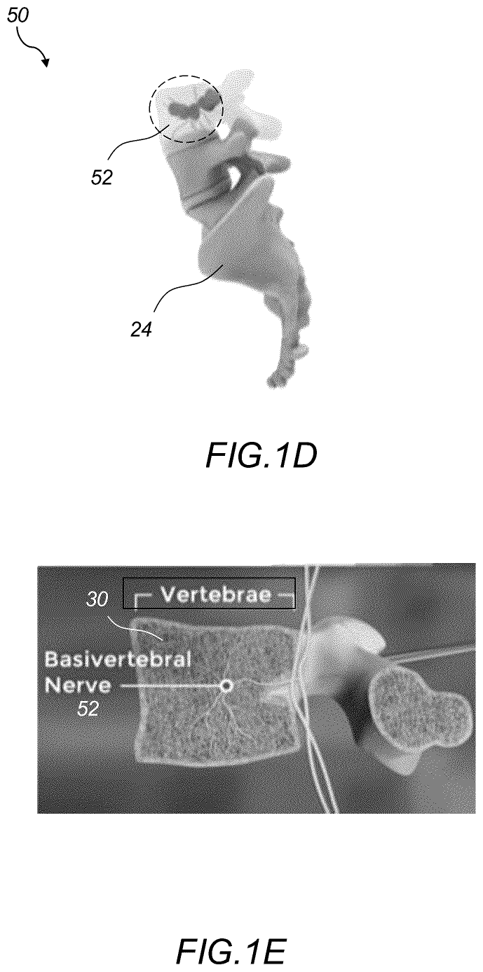

### DETAILED DESCRIPTION OF THE INVENTION

The present invention relates to devices and methods for BVN removal, and in particular, to devices, instruments, and methods that limit the size of the incision, and provide safe and accurate targeting and removal of the BVN.

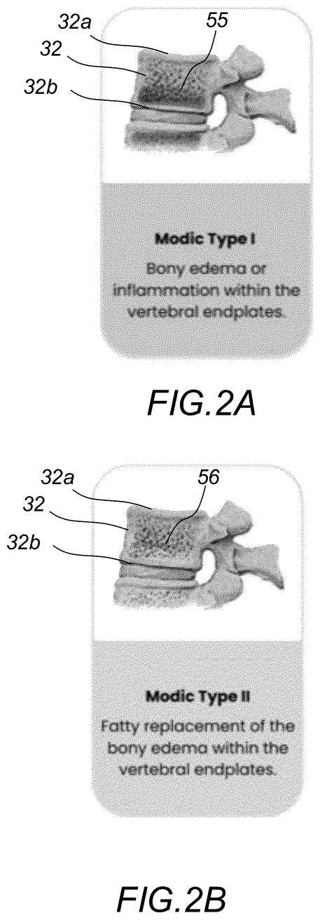

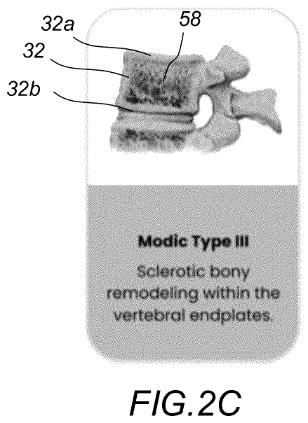

Referring to FIG. 2A-FIG. 2C pathologies of the vertebral endplates include Modic I, Modic II and Modic III types. Modic type endplate changes represent a classification for vertebral body endplate MRI signal changes that is widely recognized by radiologists and physicians of the spine. As was mentioned above, Modic type changes are believed to be associated with low back pain and are thought to be a results of normal degenerative processes, injury, inflammation, infection, or autoimmune conditions.

Modic type I pathologies include bony edema 55, bone marrow edema, and inflammation within the vertebral endplates. They are believed to be the result of fissuring of the vertebral endplates with the development of vascular granulation tissue adjacent to the endplates. Modic type II pathologies include fatty replacement 56 of the bony edema within the vertebral endplates. Modic type III pathologies include sclerotic bony remodeling 58 within the endplates.

As was mentioned above, in some of these Modic type pathologies of the vertebral endplates, basiverterbal nerve (BVN) removal is performed to relieve the patient of discomfort. Safe and accurate targeting of the BVN is critical to the success of the BVN removal procedure. The procedure is guided via fluoroscopy or computerized tomography (CT). A combination of anterior-posterior (AP), lateral, and oblique fluoroscopic views are utilized to localize the pedicle and to guide the positioning of instruments.

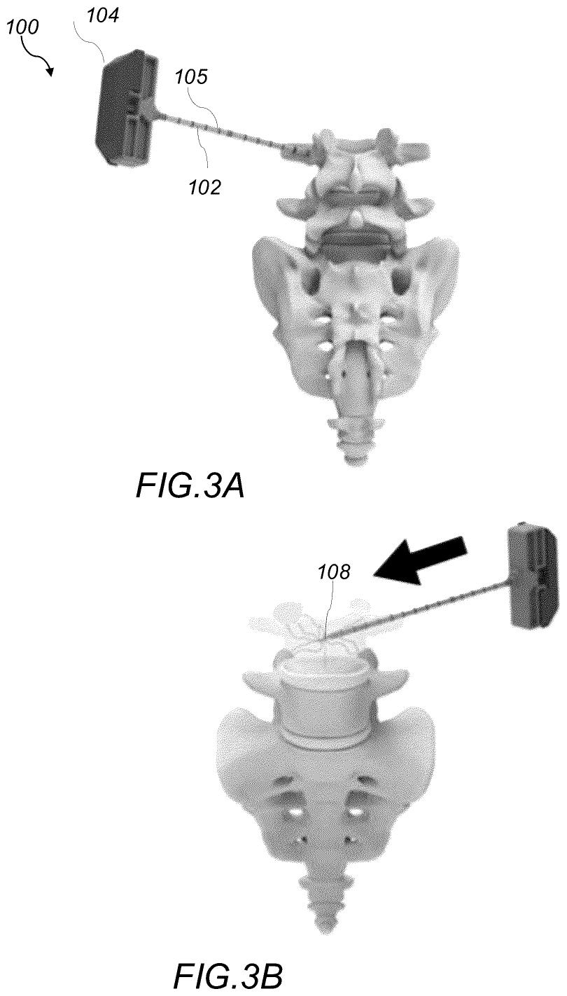

In addition to a CT or fluoroscopy imaging, equipment for BVN removal include a cannulated bevel tipped bone needle 100 which is used to access the vertebral body through a transpedicular approach. Referring to FIG. 3A-FIG. 12B, bone needle 100 includes an elongated shaft 102 having a handle 104 at a proximal end, a bevel tipped distal end 107 and an inner cannulation (cannula) 106 that allows for multiple instruments to be inserted through the bone needle and to access the vertebral body. Bone needle 100 also includes depth markings 105 on the outer surface of the shaft 102. A removable inner stylet 108 passes through the inner cannulation 106 and exits through the distal end of the shaft 102.

In one example, bone needle 100 is a single use instrument and bone needle shaft 102 is made of stainless steel. In one example, the inner cannulation has a 3 mm diameter. The bone needle 100 is designed to be advanced through the vertebra using fluoroscopy to determine the correct trajectory and depth. The BVN removal equipment further include a drill 110 or an awl used to create a channel in the vertebral body to the BVN terminus (target site) and to remove the base of the BVN, a cup curette 120 used to remove the BVN, a pituitary rongeur 130 used to evacuate the resected portions of the BVN, an ablation probe 140 used to ablate the remaining base of the BVN, a nerve monitoring probe 150 used to monitor the nerves, a nerve monitoring unit 160, and a bone graft syringe 170 used to deliver bone graft 180 to the sites of the created voids. In one example, drill 110 is a single use instrument, is made of stainless steel and has an outer diameter of 3 mm. Drill 110 can be used under power or manually with a standard T-handle 104. The depth of the drill 110 is confirmed via fluoroscopy. The cup curette 120 and the pituitary rongeur 130 are made of stainless steel and are designed to fit through the 3 mm bone needle cannulation. The ablation probe 140 is connected to a RF generator that generates electric current at the tip of the probe. The ablation probe 140 is used to cauterize the base of the BVN and to create a lesion 142. The ablation probe 140 is a single use instrument and the generator is a reuseable electrical device. Similarly, the nerve monitoring probe 150 is a single use instrument and the nerve monitoring unit 160 is a reuseable electrical device. Examples, of the bone graft 180 used with the bone graft syringe include autographs, allographs, xenografts, and synthetic bone substitutes. In one example, the bone graft is a nano-fuse bioactive glass bone graft that includes 45S5 bioactive glass with demineralized bone matrix (DBM), both coated with gelatin. The nano-fuse bioactive glass bone graft strengthens the vertebral body and contributes to the formation of new bone in the vertebral body. Instead of a drill or an awl any other hole making tool may be used.

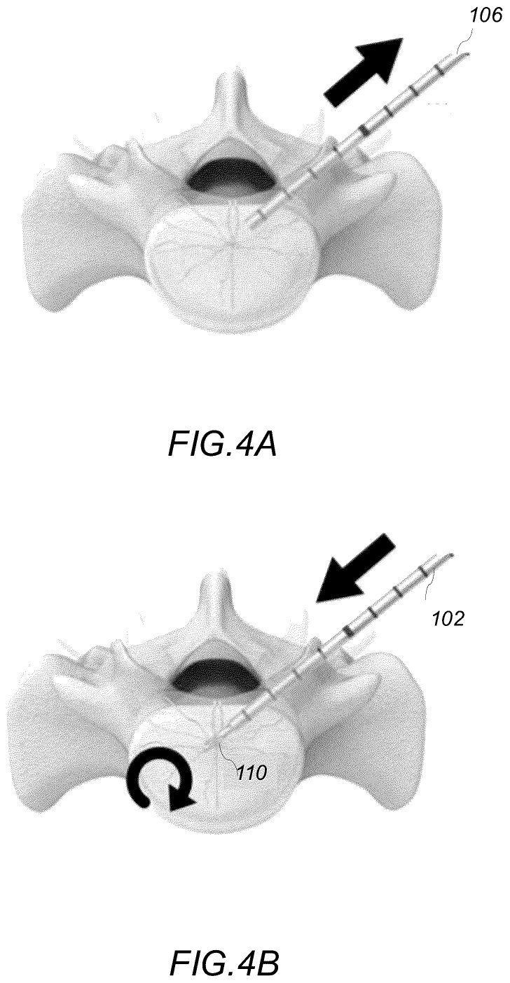

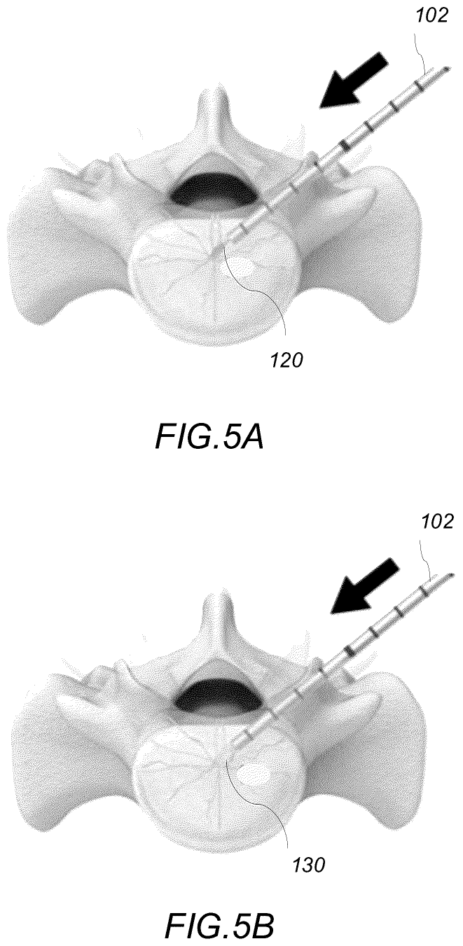

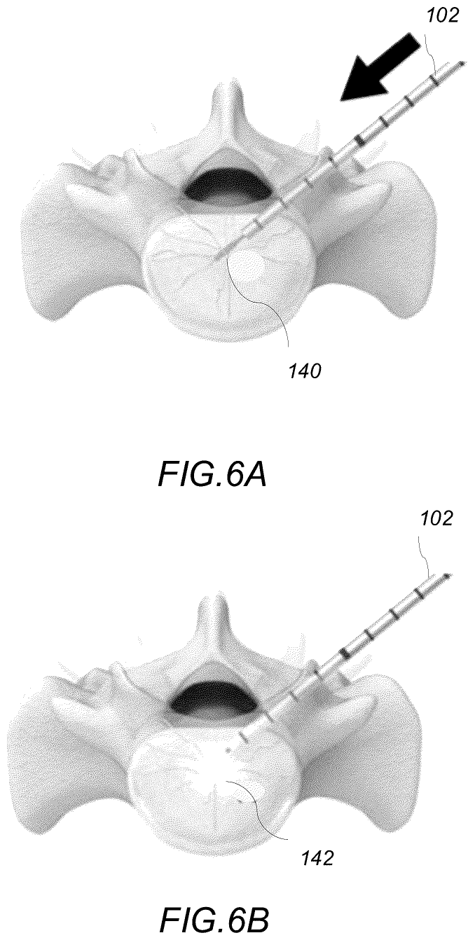

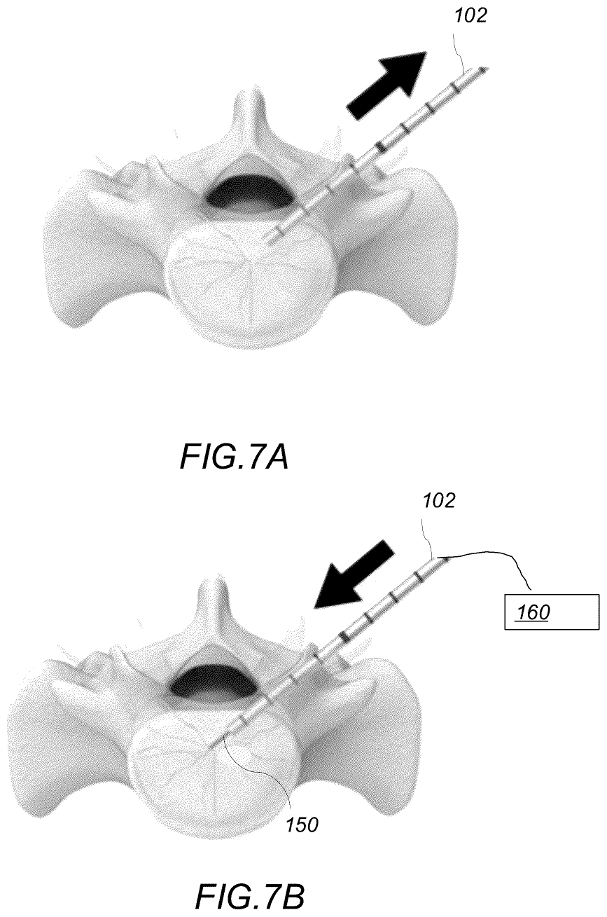

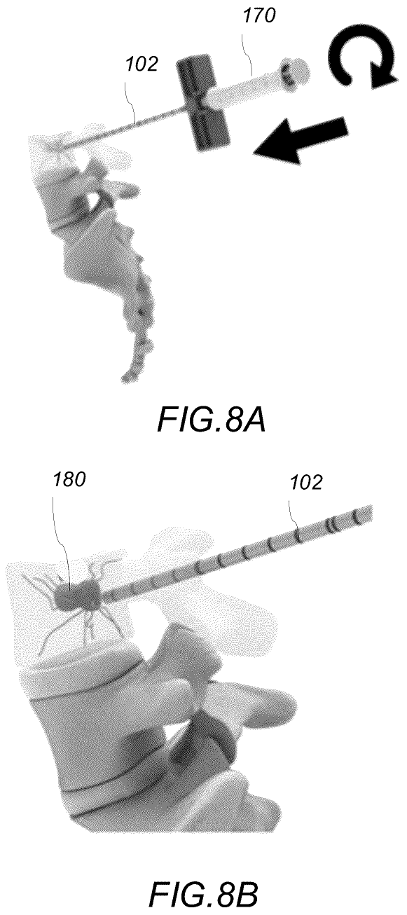

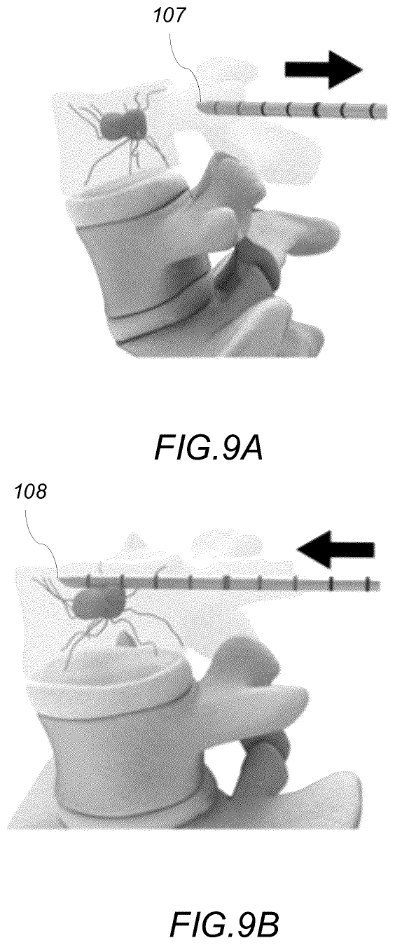

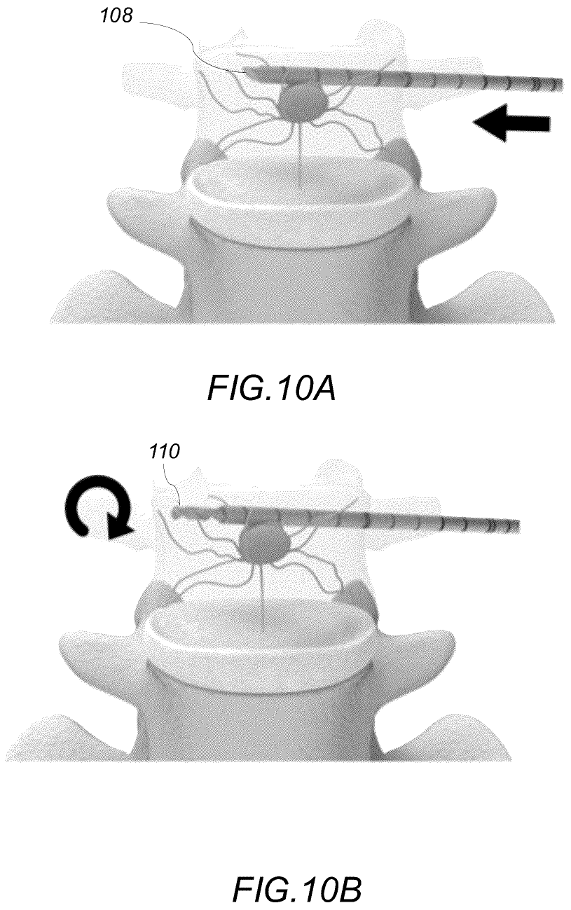

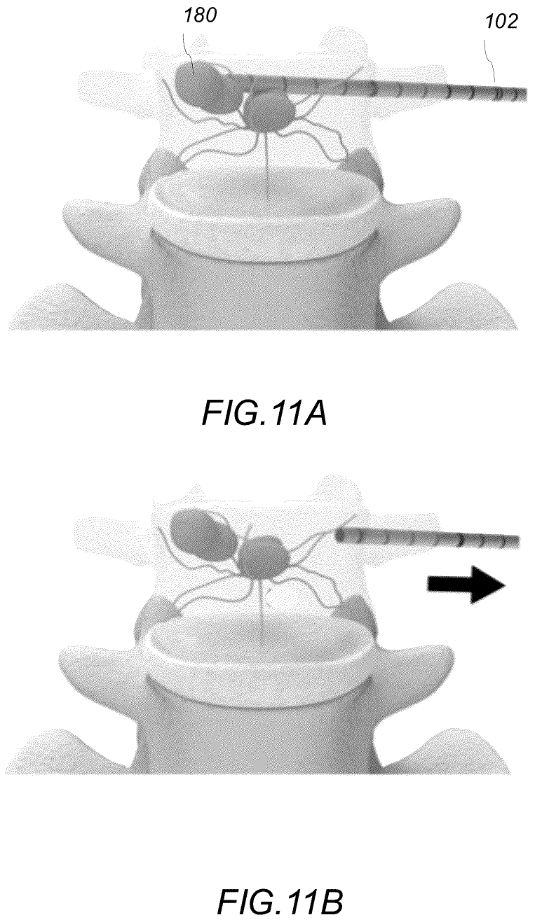

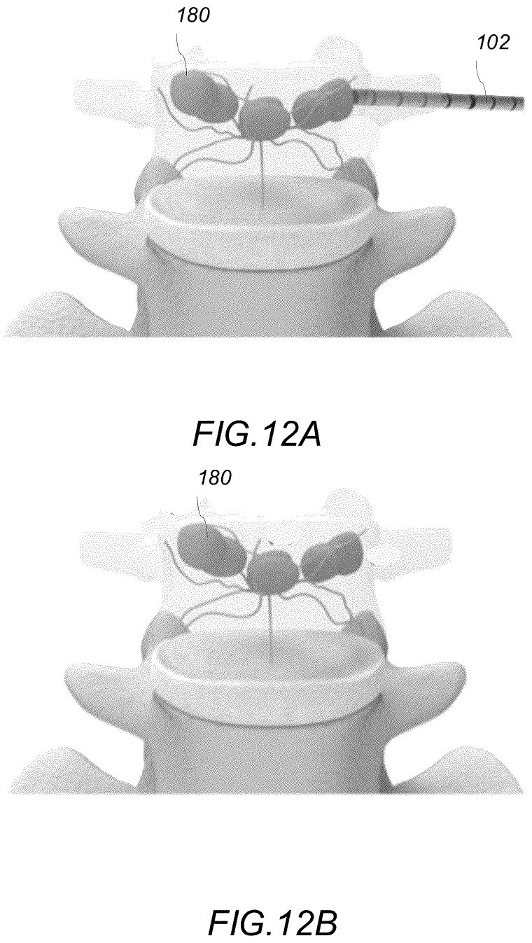

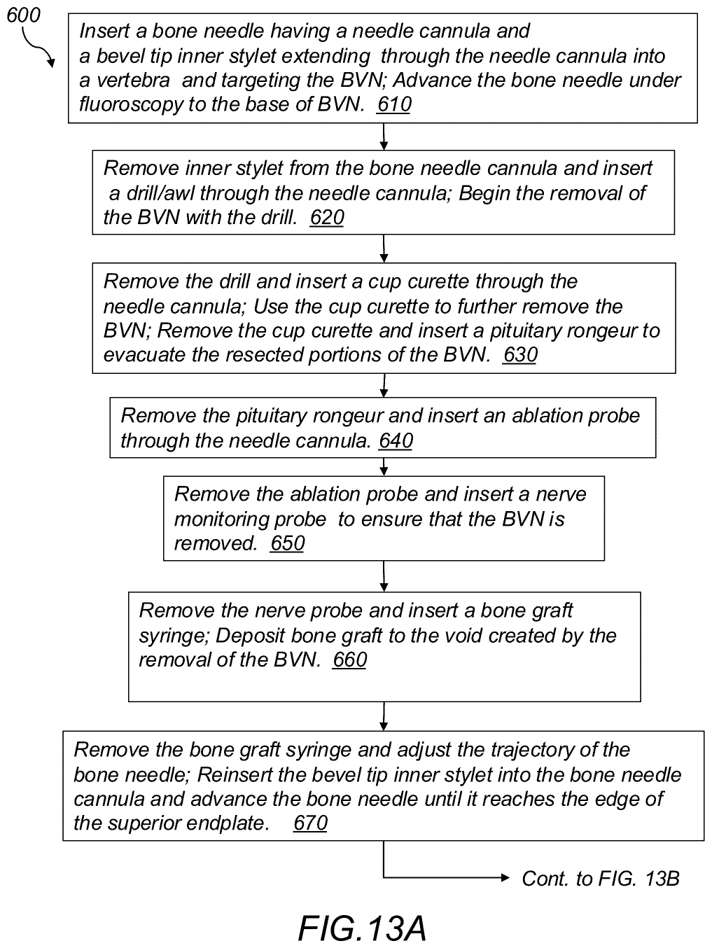

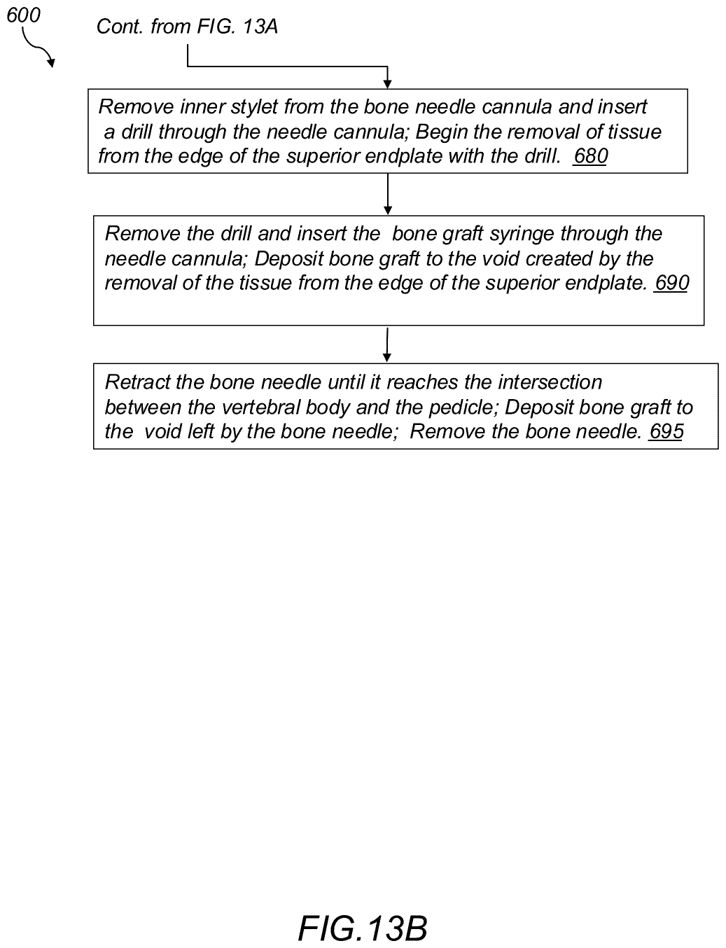

Referring to FIG. 3A-FIG. 13B, the process for BVN removal surgery 600, according to this invention includes the following steps. First, a bone needle 100 is inserted via a Wiltse transpedicular approach into the vertebral body targeting the BVN, as shown in FIG. 3A and FIG. 3B. The bone needle is advanced using fluoroscopy in order to determine the correct trajectory and depth. Bone needle 100 includes an outer needle cannula 102 and an inner stylet 108 with a bevel tipped distal end extending through the cannula. The inner stylet bevel tipped end is inserted into cannula and is advanced under fluoroscopy or CT to the base of BVN (610). Next, the inner stylet is removed from the needle cannula (shown in FIG. 4A), and drill 110 (or an awl or any other hole making tool) is inserted through the needle cannula into the BVN area (shown in FIG. 4B), and the BVN is removed (620). The depth and position of the drill is confirmed using fluoroscopy. Next, the drill 110 is removed from the needle cannula and a cup curette 120 is inserted into the needle cannula (shown in FIG. 5A), to further remove the BVN (630). The depth and position of the cup curette is confirmed using fluoroscopy. Next, the cup curette 120 is removed from the needle cannula and a pituitary rongeur 130 is inserted into the cannula (shown in FIG. 5B) to evacuate the resected portions of the BVN (630). The position of the pituitary rongeur is confirmed using fluoroscopy. Next, the pituitary rongeur 130 is removed from the needle cannula and an ablation probe 140 is inserted into the needle cannula (shown in FIG. 6A), and the remaining base of the BVN is ablated and a lesion 142 is created (shown in FIG. 6B) (640). The depth and position of the ablation probe tip is confirmed using fluoroscopy. Next, the ablation probe 140 is removed from the needle cannula (shown in FIG. 7A), and a nerve monitoring probe 150 is inserted into the needle cannula (shown in FIG. 7B), to ensure that the BVN is removed (650). The nerve monitoring probe 150 is connected to an external nerve monitoring system 160. The position of the nerve monitoring probe is confirmed using fluoroscopy. Next, the nerve monitoring probe 150 is removed from the needle cannula and a bone graft syringe 170 is connected to the proximal luer lock on the bone needle handle (shown in FIG. 8A), and bone graft 180 is filled into the void created by the removal of the BVN (shown in FIG. 8B) (660). The bone graft 180 is hydrated using a saline of the patient's blood prior to the delivery to the BVN void site. A contrast agent is added to visualize the delivery of the bone graft under fluoroscopy. The bone graft is delivered in the BVN void site by advancing the plunger in the syringe 170. Next, the bone graft syringe 170 is removed from the needle cannula handle and the trajectory of the needle cannula is adjusted so that it is directed towards the superior vertebral endplate on the contra-lateral side (shown in FIG. 9A). The inner stylet is reinserted through the needle cannula and advanced into the vertebral body until it reaches the edge of the superior vertebral endplate on the contra-lateral side (shown in FIG. 9B) (670). The position of the bone needle is confirmed using fluoroscopy. Once the correct depth is confirmed, the inner stylet 108 is removed (shown in FIG. 10A), and the drill 110 is reinserted into the needle cannula and is used to remove tissue from the edge of the superior vertebral endplate on the contra-lateral side (shown in FIG. 10B) (680). The depth and position of the drill is confirmed using fluoroscopy. Next, the drill 110 is removed from the needle cannula and the bone graft syringe 170 is connected to the proximal luer lock on the bone needle handle to deliver bone graft 180 to the void created by the removal of the tissue from the edge of the superior endplate on the contra-lateral side (shown in FIG. 11A) (690). Next, the needle cannula is retracted until it reaches the intersection between the vertebral body and the pedicle (shown in FIG. 11B), and bone graft 180 is deposited with the bone graft syringe in the void left by the needle cannula (shown in FIG. 12A). Finally, the needle cannula is removed from the surgical site and the patient is sutured (shown in FIG. 12B)(695).

Several embodiments of the present invention have been described. Nevertheless, it will be understood that various modifications may be made without departing from the spirit and scope of the invention. Accordingly, other embodiments are within the scope of the following claims.

What is claimed is:

1. A method for removing a basivertebral nerve (BVN), comprising:
inserting a bone needle into a vertebral body of a vertebra and advancing the bone needle under fluoroscopy to a BVN base site, wherein the bone needle comprises an elongated shaft having a cannula extending from a proximal end to a distal end of the shaft, and a removable bevel tipped inner stylet extending through the cannula;
removing the inner stylet from the cannula and inserting a hole making tool through the cannula into the BVN base site and once the hole making tool position and depth is confirmed via fluoroscopy removing the BVN base out with the hole making tool;
removing the hole making tool from the cannula and inserting a cup curette through the cannula into the BVN base site to further resect under fluoroscopy the BVN base;
removing the cup curette from the cannula and inserting a pituitary rongeur through the cannula into the BVN base site and removing under fluoroscopy all resected portions of the BVN base;
removing the pituitary rongeur from the cannula and inserting an electric ablation probe through the cannula into the BVN base site and once the ablation probe depth and position is confirmed ablating and cauterizing any remaining BVN base and forming a lesion in the BVN site;
removing the ablation probe from the cannula and inserting a nerve monitoring probe through the cannula into the BVN base site and testing to ensure that the BVN base has been fully removed from the BVN site and a void has been formed; and
removing the nerve monitoring probe from the cannula and attaching a syringe filled with bone graft to the proximal end of the shaft, and filling the void formed in the BVN site with bone graft.

2. The method of claim 1, further comprising:
removing the syringe and adjusting the bone needle position so that it is directed towards a superior vertebral endplate site of the vertebral body on a contra-lateral side of the vertebra;
reinserting the inner stylet into the cannula, advancing it to an edge of the superior vertebral endplate site and confirming the inner stylet position and depth with fluoroscopy;
removing the inner stylet from the cannula and reinserting the hole making tool through the cannula into the edge of the superior vertebral endplate site and once the hole making tool position and depth is confirmed via fluoroscopy removing the edge of the superior vertebral endplate out with the hole making tool;
removing any tissue fragments from the edge of the superior vertebral endplate and reattaching the syringe filled with bone graft to the proximal end of the shaft, and filling a void formed in the edge of the superior vertebral endplate site with bone graft.

3. The method of claim 2, further comprising:
retracting the bone needle till it reaches an intersection between the vertebral body and a pedicle of the vertebra and filling a void formed by the retracted bone needle in the vertebral body with bone graft.

4. The method of claim 3, further comprising removing the bone needle and suturing the bone needle insertion site.

5. The method of claim 1, wherein the hole making tool comprises a drill or an awl.

6. The method of claim 1, wherein the bone graft is hydrated using a blood saline prior to filling the BVN void.

7. The method of claim 1, wherein the bone needle further comprises a handle attached to the proximal end of the shaft and wherein the syringe is connected to a proximal luer lock on the handle.

8. The method of claim 1, further comprising adding a contrast agent to the bone graft to visualize the filling of the BVN void with bone graft under fluoroscopy.

9. The method of claim 1, wherein the bone needle further comprises depth markings on an outer surface of the elongated shaft.

10. The method of claim 1, wherein the cannula comprises a diameter of 3 mm and allows for multiple instruments to be inserted through the cannula for accessing sites in the vertebral body.

11. The method of claim 1, wherein the ablation probe is connected to a RF generator that generates electric current at a tip of the ablation probe.

12. The method of claim 1, wherein the bone needle, hole making tool, cup curette, pituitary rongeur are made of stainless steel.

13. The method of claim 1, wherein the bone graft is a nano-fuse bioactive glass bone graft that comprises 45S5 bioactive glass with demineralized bone matrix (DBM), both coated with gelatin.

14. A system for removing a basivertebral nerve (BVN), comprising:
a bone needle for inserting into a vertebral body of a vertebra and advancing under fluoroscopy to a BVN base site, wherein the bone needle comprises an elongated shaft having a cannula extending from a proximal end to a distal end of the shaft, and a removable bevel tipped inner stylet extending through the cannula;
a hole making tool for inserting through the cannula into the BVN base site and once the hole making tool position and depth is confirmed via fluoroscopy for drilling the BVN base out;
a cup curette for inserting through the cannula into the BVN base site to further resect under fluoroscopy the BVN base;
a pituitary rongeur for inserting through the cannula into the BVN base site and for removing under fluoroscopy all resected portions of the BVN base;
an electric ablation probe for inserting through the cannula into the BVN base site and once the ablation probe depth and position is confirmed for ablating and cauterizing any remaining BVN base and forming a lesion in the BVN site;
a nerve monitoring probe for inserting through the cannula into the BVN base site and for testing to ensure that the BVN base has been fully removed from the BVN site and a void has been formed; and
a syringe filled with bone graft for attaching to a proximal end of the cannula, and for filling the void formed in the BVN site with bone graft.

15. The system of claim 14 further comprising a fluoroscope and/or a CT scanner.

16. The system of claim 14, wherein the bone needle further comprises a handle attached to the proximal end of the shaft and wherein the syringe is connected to a proximal luer lock on the handle.

17. The system of claim 14, wherein the hole making tool comprises a drill or an awl.

18. The system of claim 14, further comprising a contrast agent for adding to the bone graft to visualize the filling of the BVN void with bone graft under fluoroscopy.

19. The system of claim 14, wherein the bone needle further comprises depth markings on an outer surface of the elongated shaft.

20. The system of claim 14, wherein the cannula comprises a diameter of 3 mm and allows for multiple instruments to be inserted through the cannula for accessing the vertebral body.

21. The system of claim 14, wherein the ablation probe is connected to a RF generator that generates electric current at a tip of the ablation probe.

22. The system of claim 14, wherein the bone needle, hole making tool, cup curette, pituitary rongeur are made of stainless steel.

23. The system of claim 14, wherein the bone graft is a nano-fuse bioactive glass bone graft that comprises 45S5 bioactive glass with demineralized bone matrix (DBM), both coated with gelatin.
